(19) United States
(12) Patent Application Publication
(10) Pub. No.: US 2025/0151996 A1
(43) Pub. Date: May 15, 2025

(54) ENDOSCOPE HAVING LIGHT SOURCE

(71) Applicant: InSign Medical Technology (Hong Kong) Ltd., Hong Kong (CN)

(72) Inventors: Wei-Chang Chen, Hong Kong (CN); Yuan-Shao Sung, Hong Kong (CN)

(21) Appl. No.: 18/839,560

(22) PCT Filed: Jan. 16, 2023
(86) PCT No.: PCT/CN2023/072235

§ 371 Date: Aug. 19, 2024

(30) Foreign Application Priority Data
Feb. 17, 2022 (CN) 202210148827.1

(51) Int. Cl. A61B 1/06; A61B 1/00; A61B 1/04

(52) U.S. Cl. CPC A61B 1/0684; A61B 1/00018; A61B 1/00029; A61B 1/04

(57) ABSTRACT

An endoscope having light source includes a light source assembly, a light permeable cover, a camera assembly, and functional channel. The light source assembly includes flexible substrate and light source elements on substrate. The substrate has top face, connecting, and lateral portion. The top face portion is non-parallel to and connects to the lateral portion via the connecting portion. The elements are disposed at least on lateral portion. The top face portion has apertures. The cover covers the elements. The camera assembly inside the light source assembly has one end protruded through aperture and near the cover to capture image. The functional channel inside the light source assembly has one end protruded through aperture and near the cover. In the endoscope, the elements are disposed on the flexible substrate so the problem of insufficient spaces for the light source is solved, the illuminance and the light source elements are increased.

### BACKGROUND OF THE INVENTION

### Technical Field

The present invention relates to an endoscope. More particularly, the present invention relates to an endoscope having light source.

### Description of Related Art

Currently, there are various types of endoscopes available on the market for medical, industrial, or other applications. Generally, an endoscope is required to be small in size and has sufficient illuminance brightness. When the brightness provided by the light source is insufficient, the camera will have difficulty capturing correct and effective images.

However, within the limited internal space of the endoscope, it is often unable to provide a light source with sufficient brightness. In addition to the insufficient number of light sources, the angle at which the light source can emit light is also limited. One known solution is to use an externally connected back-end light source to provide light. A light-conducting material such as optical fiber is used to guide the light into the endoscope. However, this type of back-end light source not only is bulky and lacks portability, but also subject to the issue of large power consumption. Moreover, the method of using optical fiber to emit light still has limitations in the angle of emitting light, resulting in uneven illumination and dead angles. These limitations significantly affect the development of endoscopes.

Therefore, for the aforementioned technical problems, it is necessary to provide a new technical solution for the light sources used in endoscopes to overcome the aforementioned shortcomings.

### SUMMARY

In view of the above-mentioned problems, the present invention is to provide an endoscope having light source. The light source elements are disposed on the flexible substrate, hence increasing the illumination and the number of the light source elements.

According to one aspect of the invention, an endoscope having light source is provided. The endoscope includes a light source assembly, a light permeable cover, a camera assembly, and at least one functional channel. The light source assembly includes a flexible substrate and several light source elements disposed on the flexible substrate. The flexible substrate has a top face portion, a connecting portion, and a lateral portion. The top face portion is connected to the lateral portion via the connecting portion. The top face portion is non-parallel to the lateral portion. The light source elements are disposed at least on the lateral portion. The top face portion has several apertures. The light permeable cover is disposed over the light source assembly and covers the light source elements. The camera assembly is disposed inside the light source assembly with one end of the camera assembly protruded through one of the apertures and is adjacent to the light permeable cover to perform image capturing. The functional channel is disposed inside the light source assembly with one end of the functional channel protruded through another one of the apertures and is adjacent to the light permeable cover.

The endoscope having light source has the following merits:

The light source elements of the endoscope are disposed on the flexible substrate such that the light source elements can be optionally disposed at different locations of the endoscope. The problem of insufficient space for the light source elements in the compact internal space of the endoscope can be solved. The number of light source elements can be increased, and the flexibility of configuring the light source elements can also be increased. The illuminance can be enhanced and therefore is more advantageous to be used in a variety of application scenarios.

### BRIEF DESCRIPTION OF DRAWINGS

FIG. 1 is a three-dimensional diagram of an endoscope having light source according to one embodiment of the invention;

FIG. 2 is an exploded diagram of the endoscope having light source of FIG. 1;

FIG. 3 is a front view of the light source assembly of FIG. 2;

FIG. 4 is a three-dimensional diagram of the light source assembly;

FIG. 5 is a side view of the light source assembly;

FIG. 6 is a schematic diagram showing the flexible substrate of FIG. 2 in an unfolded state;

FIG. 7 is a schematic diagram of a flexible substrate in the unfolded state according to another embodiment of the invention;

FIG. 8 is a three-dimensional diagram of the assembly of the members of the endoscope other than the light permeable cover;

FIG. 9 is a three-dimensional diagram of the assembly of FIG. 8 from another view angle;

FIG. 10 is a three-dimensional diagram of the light permeable cover of one embodiment of the invention;

FIG. 11 is a schematic diagram of the light permeable cover disposing outside the light source assembly;

FIG. 12 is a cross-sectional diagram of the assembly of FIG. 11 taken along line AA′;

FIG. 13 is a schematic diagram of the fixing base being coupled with the light permeable cover of FIG. 11;

FIG. 14 is a cross-sectional diagram of the endoscope corresponding to the location near the camera assembly; and

FIG. 15 is a cross-sectional diagram of the endoscope corresponding to the location of the functional channel.

REFERENCE NUMBERS

| | | | |
|---|---|---|---|
| endoscope | 1 | light source assembly | 11 |
| flexible substrate | 110 | flexible substrate | 110′ |
| top face portion | 111 | aperture | 111a |
| connecting portion | 112 | lateral portion | 113 |
| lateral portion | 113′ | main section | 1131 |
| main section | 1131′ | first side | 1131a |
| first side | 1131a′ | second side | 1131b |
| second side | 1131b′ | surrounding arm | 1132 |
| surrounding arm | 1132′ | protruded tab | 1133 |
| protruded tab | 1133′ | light source element | 115 |
| light permeable cover | 13 | window | 13a |
| through hole | 13b | long groove | 131 |
| slope | 131a | fixing base | 15 |
| sleeve portion | 151 | mating portion | 153 |
| long rib | 1531 | camera assembly | 16 |
| functional channel | 17 | power and/or signal cable | 18 |
| tube | 19 | cross sectional line | aa′ |
| direction | z | | |

### DETAILED DESCRIPTION

The embodiments of the invention will be detailed with accompanying drawings to elaborate the technical means of the invention which are used for achieving the desired objects of the invention. It should be understood to whom skilled in the related art that the directional terms such as up, down, left, right, front, back, or so on are for showing the spatial relations in accordance with the drawings, they are not for limiting the invention. In addition, anyone who is skilled in the art may perform multiple alternations and modifications to the embodiments without departing from the spirit and scope of the invention, and the examples and embodiments derived therefrom will still be considered falling within the scope of the invention.

In the embodiments of the invention, at least an endoscope having light source is provided. The light source elements of the endoscope are disposed on the flexible substrate such that the light source elements can be optionally disposed at different locations of the endoscope. The problem of insufficient space for the light source elements in the compact internal space of the endoscope can be solved. The number of light source elements can be increased, and the flexibility of configuring the light source elements can also be increased by disposing the light source elements at different desirable locations. The illuminance can be enhanced and can be more advantageous to be used in a variety of application scenarios.

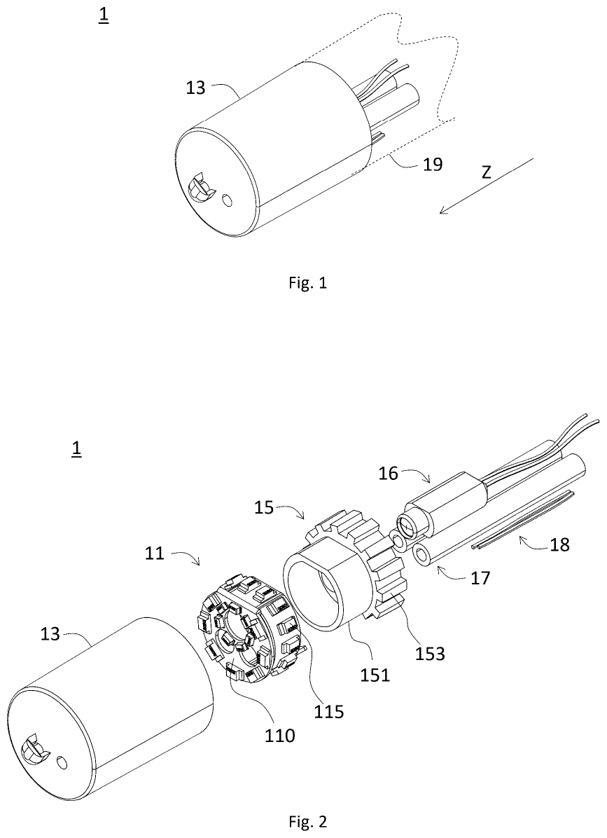

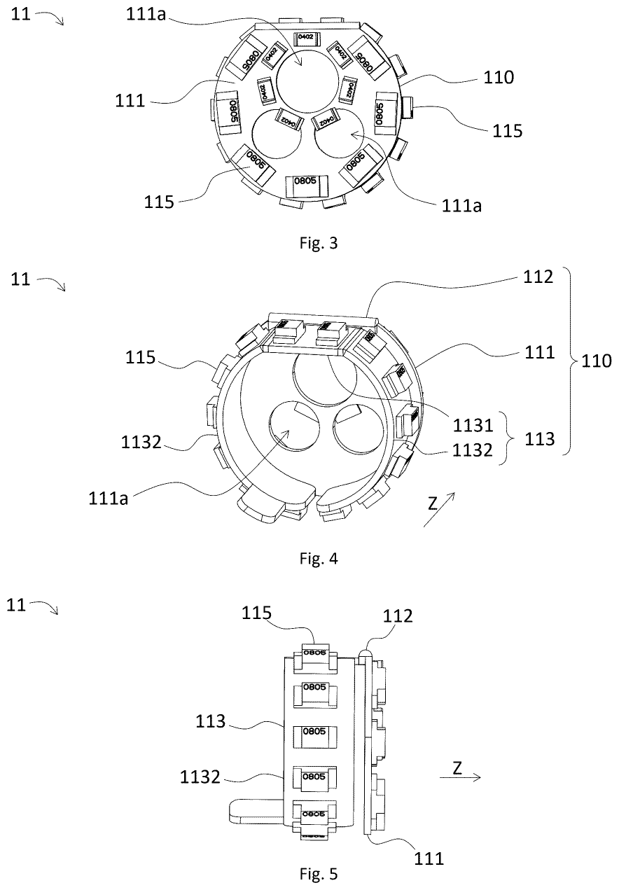

Please refer to FIG. 1 to FIG. 5. FIG. 1 is a three-dimensional diagram of an endoscope having light source according to one embodiment of the invention. FIG. 2 is an exploded diagram of the endoscope having light source of FIG. 1. FIG. 3 is a front view of the light source assembly of FIG. 2. FIG. 4 is a three-dimensional diagram of the light source assembly. FIG. 5 is a side view of the light source assembly. It should be noted that, in the present embodiment, the endoscope having light source 1 can be used for medical purposes and can also be used for industrial or environmental testing, precision machinery testing, used in manufacturing processes or other purposes. As long as there exists the needs of the capturing images in limited spaces with the light source providing enough illumination for the camera assembly to capture color, black and white, laser, infrared, or other wavelength's images, the endoscope 1 having light source is usable in all such application ranges.

The endoscope 1 having light source of the present embodiment includes a light source assembly 11, a light permeable cover 13, a camera assembly 16, and at least one functional channel 17. The light source assembly 11 includes a flexible substrate 110 and several light source elements 115 disposed on the flexible substrate 110. The flexible substrate 110 has a top face portion 111, a connecting portion 112, and a lateral portion 113. The top face portion 111 is connected to the lateral portion 113 via the connecting portion 112, and the top face portion 111 is non-parallel to the lateral portion 113. The light source elements 115 are disposed at least on the lateral portion 113, and the top face portion 111 has multiple apertures 111a. The light permeable cover 13 is disposed over the light source assembly 11 and covers the light source elements 115. The camera assembly 16 is disposed inside the light source assembly 11 with one end of the camera assembly 16 protruded through one of the apertures 111a and is adjacent to the light permeable cover 13 to perform image capturing. The functional channel 17 is disposed inside the light source assembly 11 with one end of the functional channel 17 protruded through another one of the apertures 111a and is adjacent to the light permeable cover 13.

The endoscope 1 having light source further includes a tube 19. One end of the tube 19 is coupled with the light permeable cover 13, and the other end extends to the outside of the endoscope 1. The tube 19 is bendable and is hollow on the inside so different cables or wires of the endoscope 1 can run through the tube 19. The material and the structure of the tube 19 are not limited in the present invention.

The flexible substrate 110 can be exemplified by a Flexible Printed Circuit (FPC) and has the top face portion 111, the connecting portion 112, and the lateral portion 113. In the present embodiment, the lateral portion 113 is perpendicular to the top face portion 111, as shown in FIG. 5. That is, the light source elements 115 disposed on the top face portion 111 has a light-emitting direction which is perpendicular to the light-emitting direction of the light source elements 115 disposed on the lateral portion 113, thereby achieving a lateral light emitting mechanism. In the present embodiment, the light source elements 115 are disposed both on the top face portion 11 and the lateral portion 113 of the flexible substrate 110, so the light source elements 115 emit light toward both the front (a direction Z shown in FIGS. 1, 4, & 5) and the side (a vertical direction perpendicular to the direction Z in FIGS. 1, 4 & 5) of the endoscope 1. To have the top face portion 111 and the lateral portion 113 properly bend 90 degrees, the connecting portion 112 has a U-shape curved structure as shown in FIG. 5.

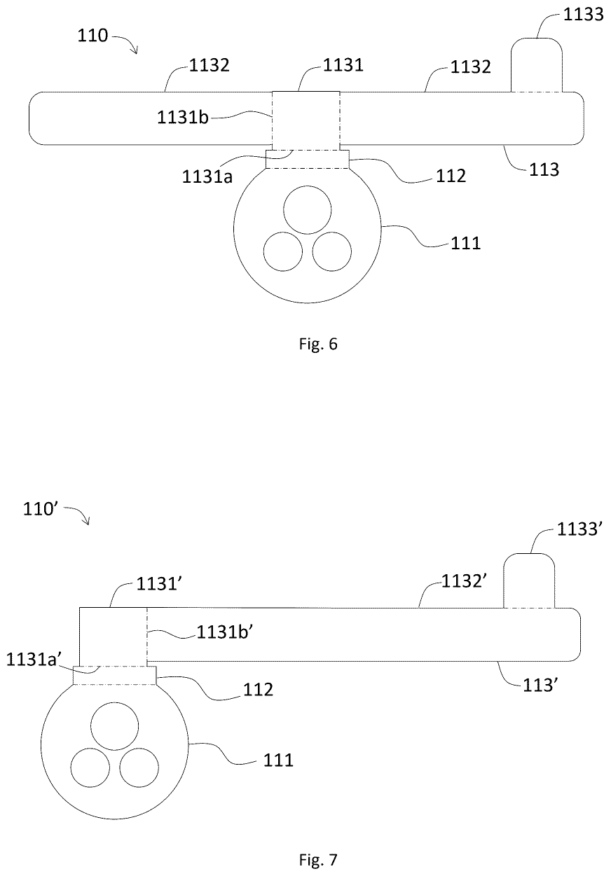

Please refer to FIG. 3 to FIG. 6. FIG. 6 is a schematic diagram showing the flexible substrate of FIG. 2 in an unfolded state. In the present embodiment, the lateral portion 113 includes a main section 1131 and a pair of surrounding arms 1132. A first side 1131a of the main section 1131 is connected to the connecting portion 112. A pair of second sides 1131b of the main section 1131 are respectively connected to the pair of surrounding arms 1132. The pair of surrounding arms 1132 extend in opposite directions and surround the top face portion 111. The light source elements 115 disposed on the pair of surrounding arms 1132 surround the top face portion 111 so a lateral emitting ring light mechanism can be achieved, as shown in FIGS. 4 & 5. The lengths of this pair of surrounding arms 1132 are not limited in the present embodiment. The length of the surrounding arm 1132 on the left can be longer than that of the surrounding arm 1132 on the right; or, in a different embodiment, the length of the surrounding arm 1132 on the left can be shorter than that of the surrounding arm 1132 on the right. The lengths of the surrounding arms 1132 can be changed based on actual needs, so long as the light source elements 115 can be arranged along the lateral side of the endoscope 1 to emit light sideways in the vertical direction perpendicular to the direction Z. The combinations of the lengths are regarded as falling within the scope of the invention.

Further, one of the surrounding arms 1132 has a protruded tab 1133 for electrically connecting to a power and/or signal cable 18 to provide power and/or signal to the light source elements 115. The protruded tab 1133 is protruded from the surrounding arm 1132 in a direction opposite to the connecting portion 112 and the top face portion 111. (The power and/or signal cable 18 is shown in FIG. 2, and the way the protruded tab 1133 and the power and/or signal cable 18 are connected is illustrated in FIG. 9.) The use of the protruded tab 1133 is beneficial to weld the power and/or signal cable 18 while assembling the endoscope 1, and the protruded tab 1133 is used as the inlet for the power and/or signal entering the flexible substrate 110. In a different embodiment, each surrounding arm 112 can be provided with one protruded tab 1133 to respectively connect to power cable and signal cable. The combinations of the power cable and/or the signal cable and one or more protruded tabs 1133 can all regarded as falling within the scope of the invention if the protruded tab(s) 1133 is used as the inlet of power and signal.

Please refer to FIG. 7, which is a schematic diagram of a flexible substrate in the unfolded state according to another embodiment of the invention. The flexible substrate 110′ of the present embodiment includes the top face portion 111, the connecting portion 112, and a lateral portion 113′. The lateral portion 113′ has a main section 1131′ and a surrounding arm 1132′. A first side 1131a′ of the main section 1131′ is connected to the connecting portion 112. A second side 1131b′ of the main section 1131′ is connected to the surrounding arm 1132′ that surrounds the top face portion 111. Only one surrounding arm 1132′ is included in this embodiment and it solely surrounds the top face portion 111. The light source elements 115 are disposed on the surrounding arm 1132′. The length of the surrounding arm 1132′ can be changed based on actual needs as well. For example, the length of the surrounding arm 1132′ can be determined based on the desired amount and density of the light source elements 115. The surrounding arm 1132′ can also have one or more protruded tab 1133′ which is protruded in the direction opposite to the connecting portion 112 and the top face portion 111 and is used for connecting to the power and/or signal cable 18. In a different embodiment, two or more protruded tabs 1133′ are disposed on the surrounding arm 1132′ to respectively connect to the power cable and the signal cable. The combinations of the power cable and/or the signal cable and one or more protruded tabs 1133′ can all regarded as falling within the scope of the invention if the protruded tab(s) 1133′ is used as the inlet of power and signal.

The light source assembly 11 according to previously mentioned embodiment, the flexible substrate 110 is the flexible printed circuit and the top face portion 111 is posed to be perpendicular to the lateral portion 113 through the curved connecting portion 112, and the top face portion 111 is surrounded by the surrounding arms 1132. The light source elements 115 disposed on the surrounding arms 1132 emit light toward the side of the endoscope 1 and form the lateral emitting ring light mechanism. In the present embodiment, LEDs are used as the light source elements 115. On the flexible substrate 110, different types of light source elements 115 can be used. For example, the light source elements 115 of visible light LEDs are disposed on the top face portion 111 and the light source elements 115 of infrared light LEDs are disposed on the lateral portion 113. In another embodiment, the infrared light LEDs are disposed on the top face portion 111 and the visible light LEDs are disposed on the lateral portion 113. In yet another embodiment, the visible light LEDs are disposed both on the top face portion 111 and the lateral portion 113; or, the infrared light LEDs are disposed both on the top face portion 111 and the lateral portion 113. The number and the arrangement of the light source elements 115 are not limited to those shown in FIGS. 2 to 5. If the light source elements 115 disposed at least on the top face portion 111 or the lateral portion 113, they can be used in the endoscope 1 of the embodiments of the invention. The room for disposing the 1o light source elements 115 is increased and the flexibility of configuring the light source elements 115 is also increased.

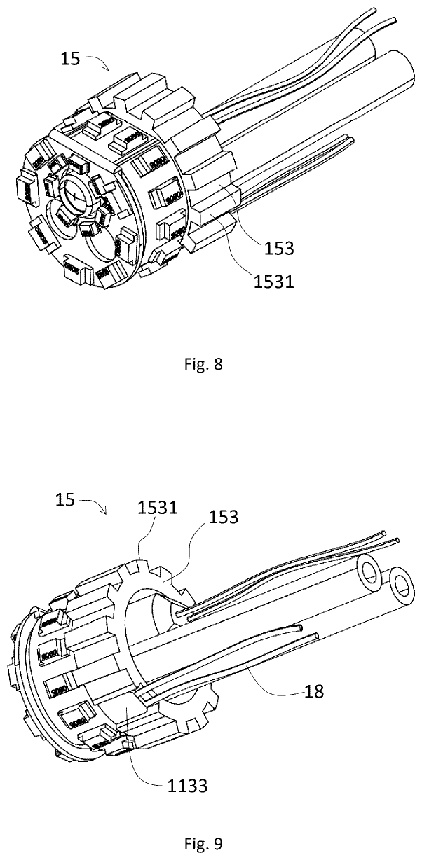

Please refer to FIGS. 2, 8, & 9. FIG. 8 is a three-dimensional diagram of the assembly of the members of the endoscope other than the light permeable cover. FIG. 9 is a three-dimensional diagram of the assembly of FIG. 8 from another view angle. The endoscope 1 having light source according to the embodiment of the invention further includes a fixing base 15. The fixing base has a sleeve portion 151. The sleeve portion 151 inserts into the light source assembly 11 which allows the top face portion 111 and the main section 1131 and the surrounding arm 1132 of the lateral portion 113 to abut against the sleeve portion 151, thereby providing support. The fixing base 15 further has a mating portion 153. The mating portion 153 is provided with several parallel long ribs 1531 which are inserted into and mated with several long grooves of the light permeable cover 13 respectively, thus allowing the fixing base 15 to be coupled with the light permeable cover 13. It is also noted that the way the protruded tab 1133 and the power and/or signal cable 18 are connected is illustrated in FIG. 9.

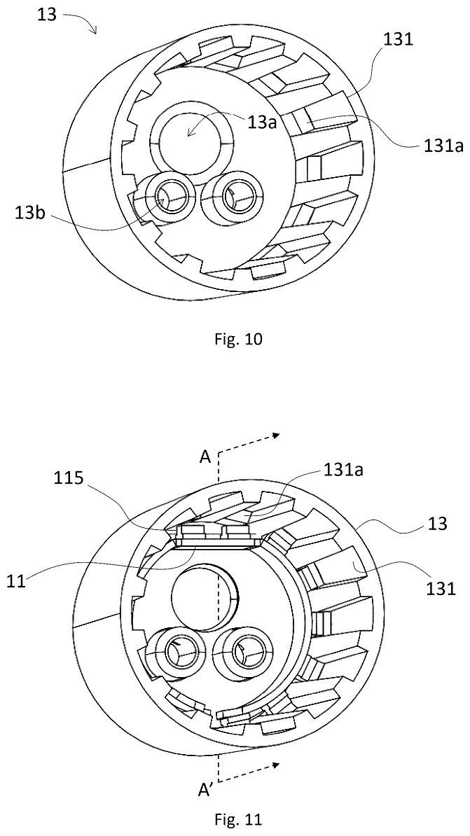

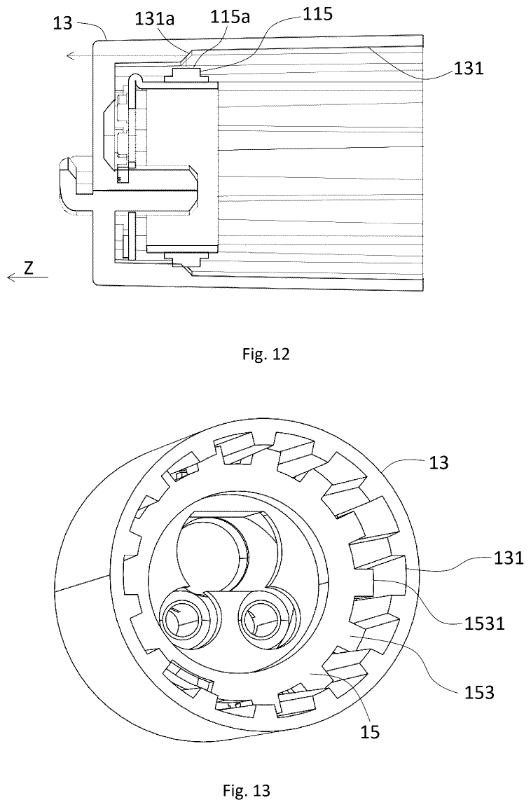

Please refer to FIG. 10 to FIG. 12. FIG. 10 is a three-dimensional diagram of the light permeable cover of one embodiment of the invention. FIG. 11 is a schematic diagram of the light permeable cover disposing outside the light source assembly. FIG. 12 is a cross-sectional diagram of the assembly of FIG. 11 taken along line AA′. The light permeable cover 13 is provided in a tube shape having one enclosed end. There are several parallel long grooves 131 arranged on the inside of the light permeable cover 13. One end of each of the long groove 131 forms a slope 131a whose location corresponds to at least one of the light source elements 151. The slope 131a and a light-emitting surface of the corresponding light source element 151 form an acute angle therebetween. When the light from the light source element 151 reaches the slope 131a, it changes its direction. Therefore, even the lateral emitting lights can be directed to move toward the front of the endoscope 1 (the dashed arrow shown in FIG. 12), which results in the increment of front illuminance of the endoscope 1. In addition to front-directing the light, in different embodiments, the slope 131a can also direct the light to travel in various lateral angles to increase the degree of light dispersion or the uniformity of sideway light emission. In the present embodiment, the slopes 131a of the long grooves 131 all have the same inclined angle; however, in different embodiments, the slopes 131a can be divided into different groups with different inclined angles. The inclined angles can be adjusted based on actual needs. In the present embodiment, the slopes 131a are equidistantly arranged on the inside lateral surface of the tube shape, to guide the light emitted from the sideway emitting light source elements 115. Therefore, a lateral light guide ring is formed and an effect of light guiding in 360 degrees can be achieved.

Please refer to FIG. 13, which is a schematic diagram of the fixing base being coupled with the light permeable cover of FIG. 11. As mentioned above, several parallel long ribs 1531 are provided on the surface of the mating portion 153 of the fixing base 15, and each is inserted into and mated with the long groove 131, hence coupling the fixing base 15 with the light permeable cover 13.

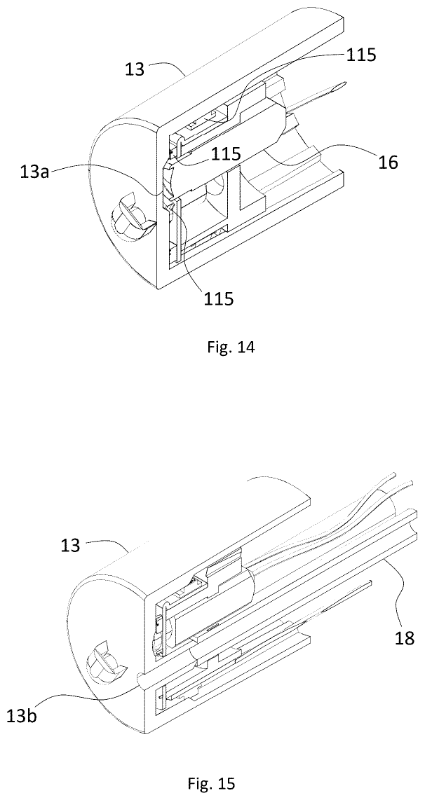

Please refer to FIG. 10, FIG. 14, and FIG. 15. FIG. 14 is a cross-sectional diagram of the endoscope corresponding to the location near the camera assembly. FIG. 15 is a cross-sectional diagram of the endoscope corresponding to the location of the functional channel. The light permeable cover 13 of the present embodiment is made of transparent or semi-transparent material and is formed in one integral piece. The light permeable cover 13 is used for having light to travel through it to perform illumination. Also, the lights from the light source elements 115 can be mixed through the light permeable cover 13 as well, increasing the uniformity of light. The light permeable cover 13 is also used to encapsulate and fix internal components. In the present embodiment, the light permeable cover 13 has a window 13a and at least one through hole 13b. The window 13a corresponds to the location of the camera assembly 16. The material thickness at the window 13a is relatively thinner, which is advantageous for the camera assembly 16 to capture images through the window 13a. The through hole 13b has a location corresponding to the functional channel 17, which allows the functional channel to communicate with outside of the light permeable cover 13. The functional channel 17 is aligned with the through hole 13b, so the functional machinery and/or functional fluids provided by back-end device pass through the functional channel 17 and the through hole 13b and extends out of the light permeable cover 13. The functional machinery used here can be medical or industrial tools such as forceps, clamps, knives, scissors, or similar tools. The functional fluids can be various gases required for medical or industrial purposes, such as inert gases or other applicable gases or liquids.

In the present embodiment, the light permeable cover 13 is exemplified by having one window 13a and two through holes 13b, and the endoscope 1 includes two functional channels 17 to match and to connected with the two through holes 13b. One of the functional channel is for providing functional machinery and the other one is for providing functional fluids.

According to the endoscope 1 of the previously mentioned embodiments elaborated with FIGS. 1 to 15, since components like the camera assembly 16 and functional channel 17 are required in the endoscope 1, a fraction of the area on the top face portion 111 of the flexible substrate 110 is needed to let these components to pass through. As a result, not the entire top face portion 111 of the flexible substrate 110 can be used to receive the light source elements 115. The number of the light source elements 115 on the top face portion 111 will have to reduce. However, since the lateral portion 113 of the flexible substrate 110 is used for disposing the light source elements 115, the phenomenon of reduced illuminance and reduced number of the light source elements 115 on the top face portion 111 can be compensated.

The endoscope having light source according to the above-mentioned embodiments are elaborated. The endoscope includes the light source assembly, the light permeable cover, the camera assembly, and at least one functional channel. The light source assembly includes the flexible substrate and several light source elements disposed on the flexible substrate. The flexible substrate has the top face portion, the connecting portion, and the lateral portion. The top face portion is connected to the lateral portion via the connecting portion. The top face portion is non-parallel to the lateral portion. The light source elements are disposed at least on the lateral portion. The top face portion has several apertures. The light permeable cover is disposed over the light source assembly and covers the light source elements. The camera assembly is disposed inside the light source assembly with one end of the camera assembly protruded through one of the apertures and is adjacent to the light permeable cover to perform image capturing. The functional channel is disposed inside the light source assembly with one end of the functional channel protruded through another one of the apertures and is adjacent to the light permeable cover. The endoscope having light source of the embodiments of the invention uses the flexible substrate to dispose the light source elements. The light source elements can be optionally disposed at different desired locations. The problem of insufficient space of disposing the light source elements can be solved. The number of light source elements can be increased, and the flexibility of configuring the light source elements can also be increased by disposing the light source elements at different desirable locations. The illuminance can be enhanced and therefore is more advantageous to be used in a variety of application scenarios.

While the present invention has been disclosed above through a number of embodiments, those embodiments are not intended to be restrictive of the scope of the invention. A person who is skilled in the art will be able to make various changes or modifications to the disclosed embodiments without departing from the spirit or scope of the invention. The scope of the patent protection sought by the applicant is defined by the appended claims.

What is claimed is:

1. An endoscope having light source, comprising:
A light source assembly comprising a flexible substrate and a plurality of light source elements disposed on the flexible substrate, the flexible substrate having a top face portion, a connecting portion, and a lateral portion, the top face portion being connected to the lateral portion via the connecting portion, wherein the top face portion is non-parallel to the lateral portion, the light source elements are disposed at least on the lateral portion, and the top face portion has a plurality of apertures;
a light permeable cover disposed over the light source assembly and covering the light source elements;
a camera assembly disposed inside the light source assembly with one end of the camera assembly protruded through one of the apertures and being adjacent to the light permeable cover to perform image capturing; and
at least one functional channel disposed inside the light source assembly with one end of the functional channel protruded through another one of the apertures and being adjacent to the light permeable cover.

2. The endoscope having light source according to claim 1, wherein the lateral portion comprises a main section and a surrounding arm, a first side of the main section is connected to the connecting portion, a second side of the main section is connected to the surrounding arm, the surrounding arm surrounds the top face portion, and the light source elements disposed on the surrounding arm surround the top face portion.

3. The endoscope having light source according to claim 2, wherein the surrounding arm has a protruded tab for electrically connecting to a power cable and/or a signal cable to provide power and/or signal to the light source elements.

4. The endoscope having light source according to claim 2 further comprising a fixing base which has a sleeve portion, wherein the sleeve portion inserts into the light source assembly which allows the top face portion and the main section and the surrounding arm of the lateral portion to abut against the sleeve portion, thereby providing support.

5. The endoscope having light source according to claim 1, wherein the lateral portion comprises a main section and a pair of surrounding arms, a first side of the main section is connected to the connecting portion, a pair of second sides are respectively connected to the pair of surrounding arms, the pair of surrounding arms extend in opposite directions and surround the top face portion, and the light source elements disposed on the pair of surrounding arms surround the top face portion.

6. The endoscope having light source according to claim 1, wherein the light permeable cover is provided in a tube shape having one enclosed end, a plurality of parallel long grooves are arranged on the inside of the light permeable cover, one end of each of the long groove forms a slope whose location corresponds to at least one of the light source element, and the slope and a light-emitting surface of the corresponding light source element form an acute angle therebetween.

7. The endoscope having light source according to claim 6 further comprising a fixing base which has a mating portion, wherein the mating portion is provided with a plurality of parallel long ribs which are inserted into and mated with the long grooves respectively allowing the fixing base to be coupled with the light permeable cover.

8. The endoscope having light source according to claim 1, wherein the lateral portion is perpendicular to the top face portion.

9. The endoscope having light source according to claim 1, wherein the connecting portion has a U-shape curved structure.

10. The endoscope having light source according to claim 1, wherein the light permeable cover has at least one through hole whose location corresponds to the functional channel which allows the functional channel to communicate with outside of the light permeable cover.
